(19) United States
(12) Patent Application Publication
(10) Pub. No.: US 2022/0076167 A1
(43) Pub. Date: Mar. 10, 2022

(54) METHOD FOR MODEL DEPLOYMENT, TERMINAL DEVICE, AND NON-TRANSITORY COMPUTER-READABLE STORAGE MEDIUM

(71) Applicant: Ping An Technology (Shenzhen) Co., Ltd., Shenzhen (CN)

(72) Inventors: Yijun TANG, Shenzhen (CN); Lan SUN, Shenzhen (CN); Liyang FAN, Shenzhen (CN)

(73) Assignee: Ping An Technology (Shenzhen) Co., Ltd., Shenzhen (CN)

(21) Appl. No.: 17/530,801

(22) Filed: Nov. 19, 2021

Related U.S. Application Data
(63) Continuation of application No. PCT/CN2012/124699, filed on Oct. 29, 2020.

(30) Foreign Application Priority Data
Sep. 9, 2020 (CN) 202010939338.9

(51) Int. Cl. G06N 20/00; G06F 16/11

(52) U.S. Cl. CPC G06N 20/00; G16H 50/20; G06F 16/116

(57) ABSTRACT

A method for model deployment, a terminal device, and a non-transitory computer-readable storage medium are provided. The method includes the following. A to-be-deployed model and an input/output description file of the to-be-deployed model are obtained. Output verification is performed on the to-be-deployed model based on the input/output description file. If the output verification of the to-be-deployed model passes, an inference service resource is determined from multiple running environments and the inference service resource is allocated to the to-be-deployed model. An inference parameter value of executing an inference service by the to-be-deployed model based on the inference service resource is determined. A resource configuration file and an inference service interface of the to-be-deployed model are generated according to the inference service resource, if the inference parameter value is greater than or equal to a preset inference parameter threshold.

### CROSS-REFERENCE TO RELATED APPLICATION(S)

The present application is a continuation under 35 U.S.C. § 120 of International Application No. PCT/CN2020/124699, filed on Oct. 29, 2020, which claims priority under 35 U.S.C. § 119(a) and/or PCT Article 8 to Chinese Patent Application No. 202010939338.9, filed on Sep. 9, 2020, the disclosures of which are hereby incorporated by reference.

### TECHNICAL FIELD

This disclosure relates to the field of artificial intelligence, and more particularly to a method for model deployment, a terminal device, and a non-transitory computer-readable storage medium.

### BACKGROUND

At present, model generation in the field of artificial intelligence (AI) generally relates to two processes: model training and model inference. The inventor found that graphics processing units (GPUs) are widely used in the model training and model inference due to powerful data processing functions of the GPU. AI models are generally developed based on several open source frameworks. Different open source frameworks and different versions of a same open source framework may not be compatible in terms of hardware level, that is, a model can run in one environment but cannot run in other environments. The inventor realized that a training model virtual environment created by using docker technology can enable the models to run compatibly in different software. However, the use of the docker technology requires configuration of very large and complete model mirroring files, and the use of the docker technology also does not solve a problem that the model cannot run when a hardware device running environment of the model is changed.

### SUMMARY

In a first aspect, implementations of the disclosure provide a method for model deployment. The method includes the following. A to-be-deployed model and an input/output description file of the to-be-deployed model are obtained. Input data is determined according to the input/output description file and output verification is performed on the to-be-deployed model based on the input/output description file and the input data. An inference service resource is determined from multiple running environments and the inference service resource is allocated to the to-be-deployed model, on condition that the output verification of the to-be-deployed model passes. An inference parameter value of executing an inference service based on the inference service resource by the to-be-deployed model is determined. A resource configuration file and an inference service interface of the to-be-deployed model are generated according to the inference service resource to complete deployment of the to-be-deployed model, if the inference parameter value is greater than or equal to a preset inference parameter threshold.

In a second aspect, implementations of the disclosure provide a terminal device. The terminal device includes a processor and a memory coupled with the processor. The memory is configured to store computer programs. The computer programs include program instructions. The processor is configured to invoke the program instructions to perform the following. A to-be-deployed model and an input/output description file of the to-be-deployed model are obtained. Input data is determined according to the input/output description file and output verification is performed on the to-be-deployed model based on the input/output description file and the input data. An inference service resource is determined from multiple running environments and the inference service resource is allocated to the to-be-deployed model, on condition that the output verification of the to-be-deployed model passes. An inference parameter value of executing an inference service based on the inference service resource by the to-be-deployed model is determined. A resource configuration file and an inference service interface of the to-be-deployed model are generated according to the inference service resource to complete deployment of the to-be-deployed model, if the inference parameter value is greater than or equal to a preset inference parameter threshold.

In a third aspect, implementations of the disclosure provide a non-transitory computer-readable storage medium. The non-transitory computer-readable storage medium stores computer programs including program instructions. The program instructions which, when executed by a processor, cause the processor to perform the following. A to-be-deployed model and an input/output description file of the to-be-deployed model are obtained. Input data is determined according to the input/output description file and output verification is performed on the to-be-deployed model based on the input/output description file and the input data. An inference service resource is determined from multiple running environments and the inference service resource is allocated to the to-be-deployed model, on condition that the output verification of the to-be-deployed model passes. An inference parameter value of executing an inference service based on the inference service resource by the to-be-deployed model is determined. A resource configuration file and an inference service interface of the to-be-deployed model are generated according to the inference service resource to complete deployment of the to-be-deployed model, if the inference parameter value is greater than or equal to a preset inference parameter threshold.

### BRIEF DESCRIPTION OF THE DRAWINGS

FIG. 1 is a schematic flow chart illustrating a method for model deployment provided in implementations of the disclosure.

FIG. 2 is a schematic flow chart illustrating performing output verification on the to-be-deployed model provided in implementations of the disclosure.

FIG. 3 is a schematic flow chart illustrating a method for model deployment provided in other implementations of the disclosure.

FIG. 4 is a schematic structural diagram illustrating an apparatus for model deployment provided in implementations of the disclosure.

FIG. 5 is a schematic structural diagram illustrating a terminal device provided in implementations of the disclosure.

### DETAILED DESCRIPTION

Technical solutions embodied in implementations of the disclosure will be described in a clear and comprehensive manner in conjunction with accompanying drawings in implementations of the disclosure. It is evident that the implementations described herein are merely some rather than all of the implementations of the disclosure. All other implementations obtained by those of ordinary skill in the art based on the implementations of the disclosure without creative efforts shall fall within the protection scope of the disclosure.

The technical solutions of the disclosure can be applied to the technical fields of artificial intelligence (AI), digital medical, blockchain, and/or mega data. In one example, information related to the disclosure, such as disease diagnosis and treatment information, can be stored in a database or a blockchain, and the disclosure is not limited thereto.

At present, using the AI technology to perform model construction based on information in a field can realize resource sharing and promote technological development in the field. For example, in the medical field, model construction based on disease diagnosis and treatment information can help people quickly understand information of a disease such as a type of the disease (i.e., a disease type), manifestation characteristics, disease causes, characteristics of patients with the disease (i.e., patient characteristics), a probability of suffering from the disease (i.e., a disease probability), diagnosis and treatment methods for the disease, and so on. For another example, model construction based on personal healthcare information can help people intuitively know health information of a group of people or the resident population in an area, such as height, weight, blood pressure, blood sugar, blood lipids, and so on. For yet another example, model construction based on medical facility information can help people quickly know the allocation of medical resources in a place, treatment conditions for a disease, and so on. As can be seen, model construction and using the model to conduct inference can be widely used. In implementations of the disclosure, model construction based on disease diagnosis and treatment information in the medical field is taken as an example for illustration. The model construction in other fields or the model construction based on other information in the medical field is the same as that provided in the implementations of the disclosure, which are not repeated herein.

The model construction based on disease diagnosis and treatment information in the medical field is taken as an example for illustration. The disease diagnosis and treatment information includes, but is not limited to, disease types, manifestation characteristics, disease causes, patient characteristics, disease probabilities, diagnosis and treatment methods, and the like. For the convenience of description, in the disclosure, the disease diagnosis and treatment information for model construction merely include four types of information: the disease types, the manifestation characteristics, basic patient characteristics, and the disease probabilities. The model construction is conducted based on the disease diagnosis and treatment information as follows. Pathological information of a disease (such as heart disease) is obtained, and types of the heart disease and detailed classification of the heart disease are determined. Each type of heart disease (i.e., heart disease type) is associated with manifestation characteristics of the heart disease of the type and characteristics of patients with the heart disease of the type. The manifestation characteristics of the heart disease include, but are not limited to, degree of angina pectoris (severe pain, mild pain, or no pain), venous pressure, a resting heart rate, a maximum heart rate, a frequency of the attack of angina pectoris, and the like. The basic characteristics of patients with the heart disease (i.e., heart disease patient characteristics) include, but are not limited to, age, gender, permanent residence area, eating habits, smoking or not, drinking or not, and the like. Thereafter, a heart disease diagnosis and treatment model can be constructed and the heart disease diagnosis and treatment model is trained with training samples. When an input sample includes one or more characteristics in the manifestation characteristics and the heart disease patient characteristics, a type of heart disease that the input sample may suffer from and a probability that the input sample may suffer from the heart disease of the type can be calculated through the model. After the heart disease diagnosis and treatment model is obtained, the heart disease diagnosis and treatment model can be deployed to an autonomous diagnosis platform.

The heart disease diagnosis and treatment model is deployed to the autonomous diagnosis platform as follows. The heart disease diagnosis and treatment model and an input/output description file of the heart disease diagnosis and treatment model are obtained. When data (e.g., age: xx, gender: x, resting heart rate: xx, maximum heart rate: xxx, degree of angina pectoris: xxxx, frequency of the attack of angina pectoris: xx) are inputted into the heart disease diagnosis and treatment model according to an input format described in the input/output description file, output data can be obtained. If a format of the output data matches an output data format (e.g., disease type: xx, disease probability: xx) specified in the input/output description file, it can be determined that output verification of the heart disease diagnosis and treatment model passes and the heart disease diagnosis and treatment model can be deployed to the autonomous diagnosis platform. The autonomous diagnosis platform includes multiple running environments, for example, the autonomous diagnosis platform includes multiple graphics processing units (GPU) and multiple GPU running schemes that can be used for model inference. A running environment is selected from the multiple running environments, for example, a GPU with a video memory of 8 gigabyte (GB, G) is used to run the heart disease diagnosis and treatment model by using 8 threads to conduct inference on ten pieces of input data, to obtain a required inference time. According to the inference time, an inference speed of the heart disease diagnosis and treatment model is determined, and the inference speed can be determined as an inference parameter value. If the inference speed is higher than a preset threshold, it means that the heart disease diagnosis and treatment model can conduct inference in the running environment. Therefore, a GPU configuration corresponding to the running environment can be stored and an interface for the autonomous diagnosis platform to invoke the heart disease diagnosis and treatment model to conduct inference can be generated, to complete the deployment of the heart disease diagnosis and treatment model on the autonomous diagnostic platform.

In implementations of the disclosure, for convenience of description, the method and apparatus for model deployment provided in the implementations of the disclosure will be described below by taking the heart disease diagnosis and treatment model as an example of the to-be-deployed model.

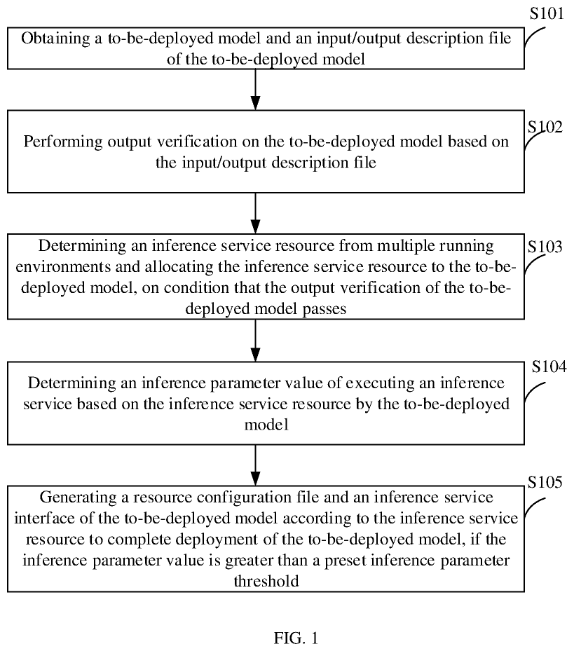

FIG. 1 is a schematic flow chart illustrating a method for model deployment provided in implementations of the disclosure. As illustrated in FIG. 1, the method provided in implementations of the disclosure includes the following. A to-be-deployed model and an input/output description file of the to-be-deployed model are obtained. Output verification is performed on the to-be-deployed model based on the input/output description file. If the output verification of the to-be-deployed model passes, an inference service resource is determined from multiple running environments and then allocated to the to-be-deployed model. An inference parameter value of executing an inference service based on the inference service resource by the to-be-deployed model is determined. If the inference parameter value is greater than a preset threshold, a resource configuration file and an inference service interface of the to-be-deployed model are generated to complete deployment of the to-be-deployed model. For convenience of description, the method provided in implementations of the disclosure will be described by taking the deployment of a heart disease diagnosis and treatment model on an autonomous diagnosis platform as an example.

The method provided in the implementations of the disclosure includes the following.

At S101, the to-be-deployed model and the input/output description file of the to-be-deployed model are obtained.

In some implementations, the input/output description file of the to-be-deployed model is obtained. The input/output description file may include an input node used for verifying the feasibility of the to-be-deployed model, an input data format corresponding to the input node, an output node for obtaining target output data, and an output data format corresponding to the output node. As an example, an input/output description file of the heart disease diagnosis and treatment model is obtained, and then according to the input/output description file of the heart disease diagnosis and treatment model, an input node (a node used for inputting manifestation characteristics), an input data format (resting heart rate: xx, maximum heart rate: xxx, degree of angina pectoris: xxxx, frequency of the attack of angina pectoris: xx), an output node (a node for outputting a probability of suffering from a heart disease), and an output data format (e.g., disease probability: xx) are obtained. Alternatively, according to the input/output description file of the heart disease diagnosis and treatment model, an input node (a node used for jointly inputting manifestation characteristics and basic patient characteristics), an input data format (resting heart rate: xx, maximum heart rate: xxx, degree of angina pectoris: xxxx, frequency of the attack of angina pectoris: xx, age: xx, gender: x, smoking or not: x, drinking or not: x), an output node (a node for jointly outputting a possible heart disease type (i.e., a type of heart disease that a patient may suffer from) and a probability of suffering from the heart disease of the type), and an output data format (disease type: xx, disease probability: xx) are obtained. The input/output description file can be determined according to actual scenarios, and the disclosure is not limited thereto.

At S102, output verification is performed on the to-be-deployed model based on the input/output description file.

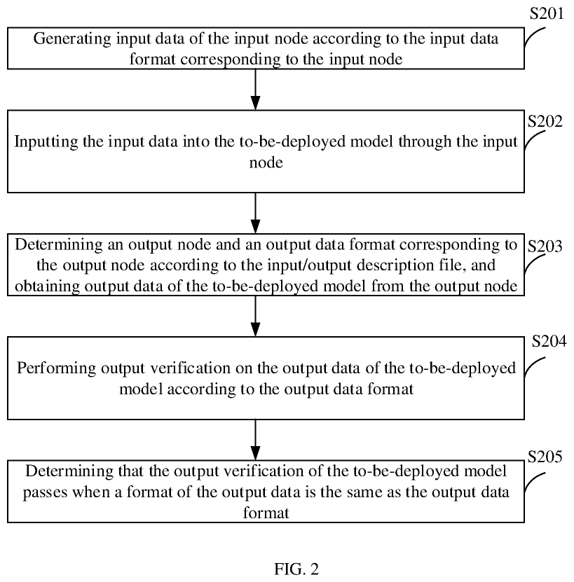

FIG. 2 is a schematic flow chart illustrating performing output verification on the to-be-deployed model provided in implementations of the disclosure. As illustrated in FIG. 2, the method for performing output verification on the to-be-deployed model may include following implementations described at S201 to S205.

At S201, input data of the input node is generated according to the input data format corresponding to the input node.

In some implementations, the input node and the input data format corresponding to the input node can be determined according to the input/output description file of the to-be-deployed model, and then the input data can be generated according to the input data format. For example, the input/output description file of the heart disease diagnosis and treatment model is obtained, and then according to the input/output description file, the input node (the node used for inputting manifestation characteristics), and the input data format (resting heart rate: xx, maximum heart rate: xxx, degree of angina pectoris: xxxx, frequency of the attack of angina pectoris: xx) are obtained, such that the input data (e.g., resting heart rate: 50, maximum heart rate: 120, degree of angina pectoris: mild pain, frequency of the attack of angina pectoris: occasionally) can be generated. Alternatively, according to the input/output description file, the input node (node used for jointly inputting manifestation characteristics and basic patient characteristics), and the input data format (resting heart rate: xx, maximum heart rate: xxx, degree of angina pectoris: xxxx, frequency of the attack of angina pectoris: xx, age: xx, gender: x, smoking or not: x, drinking or not: x) are obtained, and then the input data (e.g., resting heart rate: 50, maximum heart rate: 120, degree of angina pectoris: mild pain, frequency of the attack of angina pectoris: occasionally, age: 38, gender: male, smoking or not: no, drinking or not: no) can be generated. The input data can be determined according to actual scenarios. The disclosure is not limited thereto.

The input data can be automatically simulated and generated by the autonomous diagnosis platform according to a corresponding input data format. Alternatively, the input data can be obtained from a database (e.g., a database of the autonomous diagnosis platform or a database shared by other platforms through Internet) by the autonomous diagnosis platform according to a corresponding input data format. By performing semantically identification on each item in the input data format or determining, according to code note of each item in the input data format, semantics of input data corresponding to the item that needs to be generated, data of corresponding category can be determined as the input data.

At S202, the input data is inputted into the to-be-deployed model through the input node.

At S203, an output node and an output data format corresponding to the output node are determined according to the input/output description file, and output data of the to-be-deployed model is obtained from the output node.

In some implementations, the input data is inputted to the to-be-deployed model from the input node, the output node and the output data format corresponding to the output node are determined according to the input/output description file of the to-be-deployed model, and output verification is performed on the output data of the to-be-deployed model according to the output data format. For example, when the input data (e.g., resting heart rate: 50, maximum heart rate: 120, degree of angina pectoris: mild pain, frequency of the attack of angina pectoris: occasionally) is inputted into the heart disease diagnosis and treatment model from the node used for inputting manifestation characteristics, the output data obtained from the node for outputting a probability of suffering from heart disease may be “disease probability: 5%”. Alternatively, when the input data (e.g., resting heart rate: 50, maximum heart rate: 120, degree of angina pectoris: mild pain, frequency of the attack of angina pectoris: occasionally, age: 38, gender: male, smoking or not: no, drinking or not: no) is inputted into the heart disease diagnosis and treatment model from the node used for jointly inputting manifestation characteristics and basic patient characteristics, the output data obtained from the node for jointly outputting a possible heart disease type and a probability of suffering from the heart disease of the type may be “disease type: rheumatic heart disease, disease probability: 3%.

At S204, output verification is performed on the output data of the to-be-deployed model according to the output data format.

At S205, determine that the output verification of the to-be-deployed model passes if a format of the output data is the same as the output data format.

In some implementations, if the output data obtained from the node for outputting a probability of suffering from heart disease is “disease probability: FFFF”, and FFFF represents scrambled numbers and characters or a value of the FFFF is greater than 1, it can be determined that the format of the output data does not meet the output data format (“disease probability: xx”). That is, the output verification of the to-be-deployed model fails to pass. In other words, the heart disease diagnosis and treatment model cannot obtain correct output data on the autonomous diagnosis platform. As another example, if the output data obtained from the node for outputting a probability of suffering from heart disease is “disease probability: 5%”, it means that the format of the output data meets the output data format (because the probability of suffering from the disease is greater than or equal to zero and less than one). That is, the output verification of the to-be-deployed model passes. In other words, the heart disease diagnosis and treatment model can obtain correct output data on the autonomous diagnosis platform.

In implementations of the disclosure, the input data is determined according to the input/output description file of the to-be-deployed model, and then output verification is performed on the to-be-deployed model based on the input/output description file and the input data. In this way, it is possible to determine the feasibility of the to-be-deployed model before an inference service resource is allocated, so as to ensure that the to-be-deployed model can run normally and obtain correct model output, which can avoid a case that the to-be-deployed model cannot run or generate errors before the to-be-deployed model executes an inference service, thereby improving deployment efficiency of the to-be-deployed model.

At S103, an inference service resource is determined from multiple running environments and the inference service resource is allocated to the to-be-deployed model, when the output verification of the to-be-deployed model passes.

In some implementations, the autonomous diagnosis platform includes multiple running environments, for example, the autonomous diagnosis platform includes multiple GPUs and multiple GPU running schemes that can be used for model inference. As an example, the autonomous diagnosis platform may include multiple GPUs with different models and different operating parameters for model inference. A running environment is selected from the multiple running environments, for example, a single-core GPU with a video memory of 8G is selected to run the heart disease diagnosis and treatment model by using 8 threads, or a multi-core GPU with a video memory of 16G is selected to run the heart disease diagnosis and treatment model by using 16 threads. In addition, an inference accuracy of the GPU can be set to be F16 (a lower inference accuracy) or FP32 (a higher inference accuracy).

At S104, an inference parameter value of executing an inference service based on the inference service resource by the to-be-deployed model is determined.

In some implementations, after a running environment is selected to run the to-be-deployed model, for example, after a single-core GPU with a video memory of 8G and inference accuracy of F16 is selected to run the heart disease diagnosis and treatment model by using 8 threads or a multi-core GPU with a video memory of 16G and inference accuracy of FP32 is selected to run the heart disease diagnosis and treatment model by using 16 threads, the heart disease diagnosis and treatment model can be used to conduct inference on ten pieces of input data, and then an inference time required for the ten pieces of input data can be obtained. According to the inference time, an inference speed of the to-be-deployed model is determined, and then the inference speed can be determined as the inference parameter value. The inference parameter value can be determined according to actual scenarios. The inference parameter value may include one parameter indicator (such as inference speed) or multiple parameter indicators (e.g., a maximum amount of data that can be inferred in parallel within a specified inference time, and an inference speed under a specified inference accuracy). The disclosure is not limited thereto.

At S105, if the inference parameter value is greater than a preset inference parameter threshold, a resource configuration file and an inference service interface of the to-be-deployed model is generated to complete deployment of the to-be-deployed model.

In some implementations, the inference parameter value includes the inference speed. If a single-core GPU with a video memory of 8G and inference accuracy of F16 is selected to run the heart disease diagnosis and treatment model by using 8 threads to conduct inference on ten pieces of input data and the inference time obtained is 1 ms, it can be determined that the inference speed of the heart disease diagnosis and treatment model is 10 pieces/ms. In this case, the inference speed of the heart disease diagnosis and treatment model does not exceed the preset threshold (20 pieces/ms), and thus it can be determined that a current running environment does not meet the requirements of executing inference services by the to-be-deployed model and there is a need to change the running environment to allocate another inference service resource to the to-be-deployed model. As another example, if a multi-core GPU with a video memory of 16G and inference accuracy of FP32 is selected to run the heart disease diagnosis and treatment model by using 16 threads to conduct inference on ten pieces of input data and the inference time obtained is 0.25 ms, it can be determined that the inference speed of the heart disease diagnosis and treatment model is 40 pieces/ms which exceeds the preset threshold (20 pieces/ms), and thus it can be determined that the current running environment meets the requirements of executing inference services by the to-be-deployed model. Upon determining that the current running environment meets the requirements of executing inference services by the to-be-deployed model, the resource configuration file and the inference service interface of the to-be-deployed model can be generated according to the inference service resource. That is, a configuration file for using a multi-core GPU with the video memory of 16G and inference accuracy of FP32 to run the heart disease diagnosis and treatment model by using 16 threads and an invoking interface for invoking the heart disease diagnosis and treatment model to execute inference services on the autonomous diagnosis platform are generated, to complete the deployment of the above-mentioned to-be-deployed model.

In implementations of the disclosure, the input data is determined according to the input/output description file of the to-be-deployed model. The output verification is performed on the to-be-deployed model based on the input/output description file and the input data. If the output verification of the to-be-deployed model passes, the inference service resource is determined from the multiple running environments and the inference service resource is allocated to the to-be-deployed model. The inference parameter value of the to-be-deployed model executing the inference service based on the inference service resource is determined. If the inference parameter value is greater than or equal to the preset inference parameter threshold, the resource configuration file and the inference service interface of the to-be-deployed model are generated according to the inference service resource to complete deployment of the to-be-deployed model. By performing the output verification on the to-be-deployed model according to the input/output description file and the input data, it is possible to determine the feasibility of the to-be-deployed model, thereby ensuring that the to-be-deployed model can run correctly. In addition, by determining the inference service resource from the multiple running environments and allocating the inference service resource to the to-be-deployed model, it is possible to overcome the limitations of the running environment of the to-be-deployed model during execution of inference services by the to-be-deployed model, thereby improving the deployment efficiency and compatibility of the to-be-deployed model.

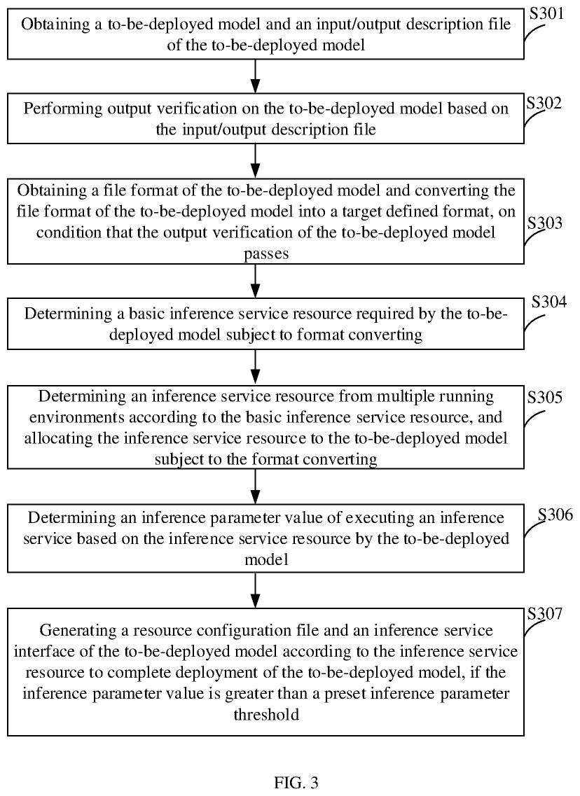

Referring to FIG. 3, FIG. 3 is a schematic flow chart illustrating a method for model deployment provided in other implementations of the disclosure.

At S301, a to-be-deployed model and an input/output description file of the to-be-deployed model are obtained.

In some implementations, the input/output description file of the to-be-deployed model is obtained. The input/output description file may include an input node used for verifying the feasibility of the to-be-deployed model, an input data format corresponding to the input node, an output node for obtaining target output data, and an output data format corresponding to the output node. As an example, an input/output description file of the heart disease diagnosis and treatment model is obtained, and then according to the input/output description file of the heart disease diagnosis and treatment model, an input node (a node used for inputting manifestation characteristics), an input data format (resting heart rate: xx, maximum heart rate: xxx, degree of angina pectoris: xxxx, frequency of the attack of angina pectoris: xx), an output node (a node for outputting a probability of suffering from a heart disease), and an output data format (e.g., disease probability: xx) are obtained. The input/output description file can be determined according to actual scenario. The disclosure is not limited thereto.

At S302, output verification is performed on the to-be-deployed model based on the input/output description file.

In some implementations, an input node and an input data format corresponding to the input node can be determined according to the input/output description file of the to-be-deployed model, and then the input data can be generated according to the input data format. The input data is inputted into the to-be-deployed model from the input node, an output node and an output data format corresponding to the output node are determined according to the input/output description file of the to-be-deployed model, and output verification is performed on the output data of the to-be-deployed model according to the output data format. For example, when the input data (e.g., resting heart rate: 50, maximum heart rate: 120, degree of angina pectoris: mild pain, frequency of the attack of angina pectoris: occasionally) is inputted into the heart disease diagnosis and treatment model from the node used for inputting manifestation characteristics, the output node (the node for outputting a probability of suffering from heart disease) and the output data format (disease probability: xx) corresponding to the output node can be determined. If the output data obtained from the node for outputting a probability of suffering from heart disease is “disease probability: 5%”, it means that the output data format of the output data is correct because the output data format is “disease probability: xx”. That is, the output verification of the to-be-deployed model passes. In other words, the heart disease diagnosis and treatment model can obtain correct output data on the autonomous diagnosis platform.

At S303, when the output verification of the to-be-deployed model passes, a file format of the to-be-deployed model is obtained, and the file format of the to-be-deployed model is converted into a target defined format.

In some implementations, the to-be-deployed model is obtained by training a target training framework. Since different target training frameworks can be used for the to-be-deployed model, the file format of the to-be-deployed model may vary. When the file format of the to-be-deployed model is different from the target defined format, the to-be-deployed model cannot run. For example, for the to-be-deployed model obtained by adopting Caffe as the target training framework, the file format of the to-be-deployed model is .pb format. However, the target defined format is .uff format because a model corresponding to the target defined format is obtained by adopting TensorFlow as the target training framework, and therefore it is necessary to convert a file in the .pb format into a file in the .uff format. Thereafter, the to-be-deployed model subject to format converting can be deployed. Since the format of the to-be-deployed model is converted into the target defined format, when the to-be-deployed model is deployed to the autonomous diagnosis platform, it is possible to overcome the limitations of the running environment of the to-be-deployed model due to inconsistent file formats during executing inference services by the to-be-deployed model, thereby improving compatibility of the to-be-deployed model.

In some implementations, the target training framework is one of Caffe, Caffeine2, TensorFlow, MxNet, CNTK, or Pytorch.

At S304, a basic inference service resource required by the to-be-deployed model subject to format converting is determined.

In some implementations, TensorRT can be used to analyze the to-be-deployed model subject to format converting, to obtain basic indicators required by execution of inference services by the to-be-deployed model, for example, to determine a basic video memory required by the to-be-deployed model. If it is determined that the basic video memory required by running of the heart disease diagnosis and treatment model is 8 GB, a GPU with a video memory of greater than 8 GB is used to run the heart disease diagnosis and treatment model for model inference, while GPUs with a video memory of less than 8 GB, such as a GPU with a video memory of 4 GB, are excluded.

At S305, an inference service resource is determined from multiple running environments according to the basic inference service resource, and the inference service resource is allocated to the to-be-deployed model.

In some implementations, the autonomous diagnosis platform includes multiple running environments, for example, the autonomous diagnosis platform includes multiple GPUs and multiple GPU running schemes that can be used for model inference. As an example, the autonomous diagnosis platform may include multiple GPUs with different models and different operating parameters for model inference. A running environment is selected from the multiple running environments, for example, a single-core GPU with a video memory of 8G is selected to run the heart disease diagnosis and treatment model by using 8 threads, or a multi-core GPU with a video memory of 16G is selected to run the heart disease diagnosis and treatment model by using 16 threads. In addition, an inference accuracy of the GPU can be set to be F16 (a lower inference accuracy) or FP32 (a higher inference accuracy).

In implementations of the disclosure, if the output verification of the to-be-deployed model passes, the file format of the to-be-deployed model is obtained, and the file format of the to-be-deployed model is converted into the target defined format. The basic inference service resource required by the to-be-deployed model subject to format converting are determined, the inference service resource is determined from the multiple running environments according to the basic inference service resource, and the inference service resource is allocated to the to-be-deployed model subject to format converting. In this way, it is possible to overcome the limitations of the running environment of the to-be-deployed model due to inconsistent file formats during execution of inference services by the to-be-deployed model, thereby improving compatibility of the to-be-deployed model.

At S306, an inference parameter value of executing an inference service based on the inference service resource by the to-be-deployed model is determined.

In some implementations, after a running environment is selected to run the to-be-deployed model, for example, after a single-core GPU with a video memory of 8G and inference accuracy of F16 is selected to run the heart disease diagnosis and treatment model by using 8 threads or a multi-core GPU with a video memory of 16G and inference accuracy of FP32 is selected to run the heart disease diagnosis and treatment model by using 16 threads, the heart disease diagnosis and treatment model can be used to conduct inference on ten pieces of input data, and then an inference time required for the ten pieces of input data can be obtained. According to the inference time, an inference speed of the to-be-deployed model is determined, and then the inference speed can be determined as the inference parameter value. The inference parameter value can be determined according to actual scenarios. The inference parameter value may include one parameter indicator (such as inference speed) or multiple parameter indicators (a maximum amount of data that can be inferred in parallel within a specified inference time, and an inference speed under a specified inference accuracy). The disclosure is not limited thereto.

At S307, if the inference parameter value is greater than a preset inference parameter threshold, a resource configuration file and an inference service interface of the to-be-deployed model is generated to complete deployment of the to-be-deployed model.

In some implementations, the inference parameter value includes the inference speed. If a single-core GPU with a video memory of 8G and inference accuracy of F16 is selected to run the heart disease diagnosis and treatment model by using 8 threads to conduct inference on ten pieces of input data and the inference time obtained is 1 ms, it can be determined that the inference speed of the heart disease diagnosis and treatment model is 10 pieces/ms. In this case, the inference speed of the heart disease diagnosis and treatment model does not exceed the preset threshold (20 pieces/ms), and thus it can be determined that a current running environment does not meet the requirements of executing inference services by the to-be-deployed model and there is a need to change the running environment to allocate another inference service resource to the to-be-deployed model. As another example, if a multi-core GPU with a video memory of 16G and inference accuracy of FP32 is selected to run the heart disease diagnosis and treatment model by using 16 threads to conduct inference on ten pieces of input data and the inference time obtained is 0.25 ms, it can be determined that the inference speed of the heart disease diagnosis and treatment model is 40 pieces/ms, which exceeds the preset threshold (20 pieces/ms), and thus it can be determined that a current running environment meets the requirements of executing inference services by the to-be-deployed model. Upon determining that the current running environment meets the requirements of executing inference services by the to-be-deployed model, resource configuration file and the inference service interface of the to-be-deployed model can be generated based on the inference service resource. That is, a configuration file for using a multi-core GPU with the video memory of 16G and inference accuracy of FP32 to run the heart disease diagnosis and treatment model by using 16 threads and an invoking interface for invoking the heart disease diagnosis and treatment model to execute inference services on the autonomous diagnosis platform are generated, to complete the deployment of the above-mentioned to-be-deployed model.

In implementations of the disclosure, if the inference parameter value is less than the preset inference parameter threshold, the method proceeds to determining the inference service resource from the multiple running environments, to determine another inference service resource that is to be allocated to the to-be-deployed model. The multiple running environments include running environments formed by changing at least one of number of graphics processing units (GPU), models of the GPUs, or GPU running schemes. In this way, it is possible to overcome the impact of the mismatched operating environment on the inference performance during executing inference services by the to-be-deployed model, thereby improving the deployment efficiency of the to-be-deployed model.

In implementations of the disclosure, if the output verification of the to-be-deployed model passes, the file format of the to-be-deployed model is obtained, and the file format of the to-be-deployed model is converted into the target defined format. The basic inference service resource required by the to-be-deployed model subject to format converting are determined, the inference service resource is determined from the multiple running environments according to the basic inference service resource, and the inference service resource is allocated to the to-be-deployed model subject to format converting. In this way, it is possible to overcome the limitations of the running environment of the to-be-deployed model due to inconsistent file formats during executing inference services by the to-be-deployed model, thereby improving compatibility of the to-be-deployed model.

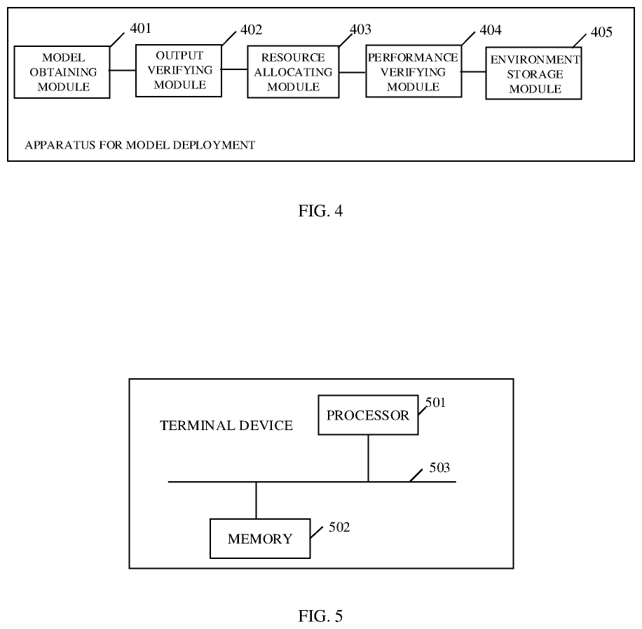

FIG. 4 is a schematic structural diagram illustrating an apparatus for model deployment provided in implementations of the disclosure.

A model obtaining module 401 is configured to obtain a to-be-deployed model and an input/output description file of the to-be-deployed model.

In some implementations, the model obtaining module 401 is configured to obtain the input/output description file of the to-be-deployed model. The input/output description file may include an input node used for verifying the feasibility of the to-be-deployed model, an input data format corresponding to the input node, an output node for obtaining target output data, and an output data format corresponding to the output node. As an example, an input/output description file of the heart disease diagnosis and treatment model is obtained, and then according to the input/output description file of the heart disease diagnosis and treatment model, an input node (a node used for inputting manifestation characteristics), an input data format (resting heart rate: xx, maximum heart rate: xxx, degree of angina pectoris: xxxx, frequency of the attack of angina pectoris: xx), an output node (a node for outputting a probability of suffering from a heart disease), and an output data format (e.g., disease probability: xx) are obtained. The input/output description file can be determined according to actual scenario. The disclosure is not limited thereto.

An output verifying module 402 is configured to determine input data according to the input/output description file and perform output verification on the to-be-deployed model based on the input/output description file and the input data.

In some implementations, the output verifying module 402 is configured to determine an input node and an input data format corresponding to the input node according to the input/output description file of the to-be-deployed model, and then generate the input data according to the input data format. The input data is inputted into the to-be-deployed model from the input node, an output node and an output data format corresponding to the output node are determined according to the input/output description file of the to-be-deployed model, and output verification is performed on the output data of the to-be-deployed model according to the output data format. For example, when the input data (e.g., resting heart rate: 50, maximum heart rate: 120, degree of angina pectoris: mild pain, frequency of the attack of angina pectoris: occasionally) is inputted into the heart disease diagnosis and treatment model from the node used for inputting manifestation characteristics, the output node (the node for outputting a probability of suffering from heart disease) and the output data format (disease probability: xx) corresponding to the output node can be determined. If the output data obtained from the node for outputting a probability of suffering from heart disease is “disease probability: 5%”, it means that the output data format of the output data is correct because the output data format is “disease probability: xx”. That is, the output verification of the to-be-deployed model passes. In other words, the heart disease diagnosis and treatment model can obtain correct output data on the autonomous diagnosis platform.

A resource allocating module 403 is configured to determine an inference service resource from multiple running environments and allocate the inference service resource to the to-be-deployed model.

In some implementations, the autonomous diagnosis platform includes multiple running environments, for example, the autonomous diagnosis platform includes multiple GPUs and multiple GPU running schemes that can be used for model inference. As an example, the autonomous diagnosis platform may include multiple GPUs with different models and different operating parameters for model inference. The resource allocating module 403 is configured to select a running environment from the multiple running environments, for example, the resource allocating module 403 is configured to select a single-core GPU with a video memory of 8G to run the heart disease diagnosis and treatment model by using 8 threads, or a multi-core GPU with a video memory of 16G to run the heart disease diagnosis and treatment model by using 16 threads. In addition, an inference accuracy of the GPU can be set to be F16 (a lower inference accuracy) or FP32 (a higher inference accuracy).

A performance verifying module 404 is configured to determine an inference parameter value of executing an inference service based on the inference service resource by the to-be-deployed model.

In some implementations, after a running environment is selected to run the to-be-deployed model, for example, after a single-core GPU with a video memory of 8G and inference accuracy of F16 is selected to run the heart disease diagnosis and treatment model by using 8 threads or a multi-core GPU with a video memory of 16G and inference accuracy of FP32 is selected to run the heart disease diagnosis and treatment model by using 16 threads, the performance verifying module 404 is configured to use the heart disease diagnosis and treatment model to conduct inference on ten pieces of input data, to obtain an inference time required for the ten pieces of input data. According to the inference time, an inference speed of the to-be-deployed model is determined, and then the inference speed can be determined as the inference parameter value. The inference parameter value can be determined according to actual scenarios. The inference parameter value may include one parameter indicator (such as inference speed), or multiple parameter indicators (an amount of data that can be inferred in parallel within a specified inference time, and an accuracy of an inference result obtained within a specified inference time). The disclosure is not limited thereto.

An environment storage module 405 is configured to generate a resource configuration file and an inference service interface of the to-be-deployed model according to the inference service resource to complete deployment of the to-be-deployed model.

In some implementations, the inference parameter value includes the inference speed. If a single-core GPU with a video memory of 8G and inference accuracy of F16 is selected to run the heart disease diagnosis and treatment model by using 8 threads to conduct inference on ten pieces of input data, and the inference time obtained is 1 ms, it can be determined that the inference speed of the heart disease diagnosis and treatment model is 10 pieces/ms. In this case, the inference speed of the heart disease diagnosis and treatment model does not exceed the preset threshold (20 pieces/ms). That is, it can be determined that a current running environment does not meet the requirements of executing inference services by the to-be-deployed model, and there is a need to change the running environment to allocate another inference service resource to the to-be-deployed model. As another example, when a multi-core GPU with a video memory of 16G and inference accuracy of FP32 is selected to run the heart disease diagnosis and treatment model by using 16 threads to conduct inference on ten pieces of input data, and the inference time obtained is 0.25 ms, it can be determined that the inference speed of the heart disease diagnosis and treatment model is 40 pieces/ms, which exceeds the preset threshold (20 pieces/ms). Therefore, it can be determined that a current running environment meets the requirements of executing inference services by the to-be-deployed model. The environment storage module 405 is configured to generate a resource configuration file and the inference service interface of the to-be-deployed model based on the inference service resource, upon determining that the current running environment meets the requirements of executing inference services by the to-be-deployed model. That is, the environment storage module 405 is configured to generate a configuration file for using a multi-core GPU with the video memory of 16G and inference accuracy of FP32 to run the heart disease diagnosis and treatment model by using 16 threads and an invoking interface for invoking the heart disease diagnosis and treatment model to execute inference services on the autonomous diagnosis platform, to complete the deployment of the above-mentioned to-be-deployed model.

In implementations of the disclosure, the input data is determined according to the input/output description file of the to-be-deployed model. The output verification is performed on the to-be-deployed model based on the input/output description file and the input data. If the output verification of the to-be-deployed model passes, the inference service resource is determined from multiple running environments and the inference service resource is allocated to the to-be-deployed model. The inference parameter value of executing an inference service based on the inference service resource by the to-be-deployed model is determined. If the inference parameter value is greater than or equal to the preset inference parameter threshold, the resource configuration file and the inference service interface of the to-be-deployed model are generated according to the inference service resource to complete deployment of the to-be-deployed model. By performing the output verification on the to-be-deployed model according to the input/output description file and input data, it is possible to determine the feasibility of the to-be-deployed model, thereby ensuring that the to-be-deployed model can run correctly. In addition, by determining the inference service resource from the multiple running environments and allocating the inference service resource to the to-be-deployed model, it is possible to overcome the limitations of the running environment of the to-be-deployed model during execution of inference services by the to-be-deployed model, thereby improving the deployment efficiency and compatibility of the to-be-deployed model.

FIG. 5 is a schematic structural diagram illustrating a terminal device provided in implementations of the disclosure. As illustrated in FIG. 5, the terminal device illustrated in FIG. 5 may include at least one processor 501 and memory 502. The processor 501 and the memory 502 are coupled to each other, for example, the processor 501 and the memory 502 are coupled to each other via a bus 503. The memory 502 is configured to store computer programs. The computer programs include program instructions. The processor 501 is configured to invoke the program instructions to perform the following operations. A to-be-deployed model and an input/output description file of the to-be-deployed model are obtained. Input data is determined according to the input/output description file and output verification is performed on the to-be-deployed model based on the input/output description file and the input data. An inference service resource is determined from multiple running environments and the inference service resource is allocated to the to-be-deployed model, on condition that the output verification of the to-be-deployed model passes. An inference parameter value of executing an inference service based on the inference service resource by the to-be-deployed model is determined. A resource configuration file and an inference service interface of the to-be-deployed model are generated according to the inference service resource to complete deployment of the to-be-deployed model, if the inference parameter value is greater than or equal to a preset inference parameter threshold.

In some implementations, the processor 501 is further configured to: determine an input node and an input data format corresponding to the input node according to the input/output description file, and generate the input data of the input node according to the input data format. The processor 501 is further configured to: input the input data into the to-be-deployed model through the input node; determine an output node and an output data format corresponding to the output node according to the input/output description file, and obtain output data of the to-be-deployed model from the output node; perform output verification on the output data of the to-be-deployed model according to the output data format; determine that the output verification of the to-be-deployed model passes if a format of the output data is the same as the output data format.

In some implementations, the processor 501 is further configured to: obtain a file format of the to-be-deployed model, and convert the file format of the to-be-deployed model into a target defined format; determine a basic inference service resource required by the to-be-deployed model subject to format converting, determine the inference service resource from the multiple running environments according to the basic inference service resource, and allocate the inference service resource to the to-be-deployed model subject to the format converting.

In some implementations, the processor 501 is further configured to: proceed to determining the inference service resource from the multiple running environments, to determine another inference service resource that is to be allocated to the to-be-deployed model, if the inference parameter value is less than the preset inference parameter threshold. The multiple running environments comprise running environments formed by changing at least one of number of graphics processing units (GPU), models of the GPUs, or GPU running schemes.

In some implementations, the to-be-deployed model is obtained by training a target training framework, where the target training framework is one of Caffe, Caffeine2, TensorFlow, MxNet, CNTK, or Pytorch.

In some implementations, training sample data for the to-be-deployed model includes at least one of disease diagnosis and treatment information, personal healthcare information, or medical facility information.

In some implementations, the processor 501 may be a central processing unit (CPU). The processor may also be other general-purpose processors, digital signal processors (DSP), application specific integrated circuits (ASIC), field-programmable gate arrays (FPGA), or other programmable logic devices, discrete gates, or transistor logic devices, discrete hardware components, or the like. The general-purpose processor may be a microprocessor or any conventional processor, or the like.

The at least one memory 502 may include a read-only memory and a random access memory, and be configured to provide instructions and data to the processor 501. The at least one memory 502 may further include a non-transitory random access memory. For example, the memory 502 may store device-type information.

In implementations, the above-mentioned terminal device can execute the implementations provided in the steps in FIGS. 1 to 3 through built-in functional modules of the terminal device. For specific details, reference may be made to the implementations provided in the above-mentioned steps, which will not be repeated herein.

In implementations of the disclosure, the input data is determined according to the input/output description file of the to-be-deployed model. The output verification is performed on the to-be-deployed model based on the input/output description file and the input data. If the output verification of the to-be-deployed model passes, the inference service resource is determined from multiple running environments and the inference service resource is allocated to the to-be-deployed model. The inference parameter value of executing an inference service based on the inference service resource by the to-be-deployed model is determined. If the inference parameter value is greater than or equal to the preset inference parameter threshold, the resource configuration file and the inference service interface of the to-be-deployed model are generated according to the inference service resource to complete deployment of the to-be-deployed model. By performing the output verification on the to-be-deployed model according to the input/output description file and input data, it is possible to determine the feasibility of the to-be-deployed model, thereby ensuring that the to-be-deployed model can run correctly. In addition, by determining the inference service resource from the multiple running environments and allocating the inference service resource to the to-be-deployed model, it is possible to overcome the limitations of the running environment of the to-be-deployed model during execution of inference services by the to-be-deployed model, thereby improving the deployment efficiency and compatibility of the to-be-deployed model.

Implementations of the disclosure provide a computer-readable storage medium. The computer-readable storage medium stores computer programs, and the computer programs include program instructions which, when executed by a processor, cause the processor to implement the method for model deployment provided in each step in FIG. 1 to FIG. 3. For specific details, reference may be made to implementations provided in the above operations, which will not be repeated herein.

In one example, the storage medium provided in implementations of the disclosure is a non-transitory computer-readable storage medium or a transitory computer-readable storage medium.

The computer-readable storage medium may be internal storage unit of the apparatus for model deployment or the terminal device provided in any of the foregoing implementations, such as the hard disk or memory of an electronic device. The computer-readable storage medium may also be an external storage device of the electronic device, such as a plug-in hard disk, a smart media card (SMC), a secure digital (SD) card, or a flash card equipped on the electronic device. In addition, the computer-readable storage medium may also include both the internal storage unit of the electronic device and the external storage device. The computer-readable storage medium is configured to store the computer programs and other programs and data required by the electronic device. The computer-readable storage medium can also be configured to temporarily store data that has been output or will be outputted.

The terms “first”, “second”, “third”, “fourth”, and the like used in the specification, the claims, and the accompany drawings of the disclosure are used to distinguish different objects rather than describe a particular order. The terms “include”, “comprise”, and “have” as well as variations thereof are intended to cover non-exclusive inclusion. For example, a process, method, system, product, or apparatus including a series of steps or units is not limited to the listed steps or units, on the contrary, it can optionally include other steps or units that are not listed; alternatively, other steps or units inherent to the process, method, product, or device can be included either. The term “implementation” referred to herein means that a particular feature, structure, or feature described in conjunction with the implementation may be contained in at least one implementation of the disclosure. The phrase appearing in various places in the specification does not necessarily refer to the same implementations, nor does it refer to an independent or alternative implementation that is mutually exclusive with other implementations. It is expressly and implicitly understood by those of ordinary skill in the art that an implementation described herein may be combined with other implementations. The term “and/or” used in the specification of the disclosure and the appended claims refers to any combination and all possible combinations of one or more of the associated listed items, and includes these combinations.

Those of ordinary skill in the art will appreciate that units and algorithmic operations of various examples described in connection with implementations herein can be implemented by electronic hardware, by computer software, or by a combination of computer software and electronic hardware. In order to clearly explain interchangeability of hardware and software, in the above description, configurations and operations of each example have been generally described according to functions. Whether these functions are performed by means of hardware or software depends on the application and the design constraints of the associated technical solution. Those of ordinary skill in the art may use different methods for teaching management with regard to each particular application to implement the described functionality, but such methods should not be regarded as lying beyond the scope of the disclosure.

The methods and related devices provided in the implementations of the disclosure are described with reference to the method flowcharts and/or structural schematic diagrams provided in the implementations of the disclosure. Specifically, each process and/or or a block in the method flowcharts and/or structural schematics, or a combination of processes and/or blocks in the flowcharts and/or block diagrams can be implemented by computer program instructions. These computer program instructions can be provided to a processor of a general-purpose computer, a special-purpose computer, an embedded processor, or other programmable data processing equipment to generate a machine, so that the instructions executed by the processor of the computer or other programmable data processing equipment can produce a device that realizes the functions specified in one block or multiple blocks in a flow chart or multiple flows and/or a schematic structural diagram. These computer program instructions can also be stored in a computer-readable memory that can guide a computer or other programmable data processing equipment to work in a specific manner, so that the instructions stored in the computer-readable memory produce an article of manufacture including the instruction device. The instruction device realizes the functions specified in one process or multiple processes in the flowchart and/or one block or multiple blocks in the schematic structural diagram. These computer program instructions can also be loaded on a computer or other programmable data processing equipment, so that a series of operation steps are executed on the computer or other programmable equipment to produce computer-implemented processing, and the instructions executed on the computer or other programmable equipment can provide steps for implementing the functions specified in one block or multiple blocks in the flow chart or the flow chart and/or the structure.

What is claimed is:

1. A method for model deployment, comprising:
obtaining a to-be-deployed model and an input/output description file of the to-be-deployed model;
determining input data according to the input/output description file and performing output verification on the to-be-deployed model based on the input/output description file and the input data;
determining an inference service resource from a plurality of running environments and allocating the inference service resource to the to-be-deployed model, on condition that the output verification of the to-be-deployed model passes;
determining an inference parameter value of executing an inference service based on the inference service resource by the to-be-deployed model; and
generating a resource configuration file and an inference service interface of the to-be-deployed model according to the inference service resource to complete deployment of the to-be-deployed model, if the inference parameter value is greater than or equal to a preset inference parameter threshold.

2. The method of claim 1, wherein determining the input data according to the input/output description file comprises:
determining an input node and an input data format corresponding to the input node according to the input/output description file; and
generating the input data of the input node according to the input data format.

3. The method of claim 2, wherein performing the output verification on the to-be-deployed model based on the input/output description file and the input data comprises:
inputting the input data into the to-be-deployed model through the input node;
determining an output node and an output data format corresponding to the output node according to the input/output description file, and obtaining output data of the to-be-deployed model from the output node;
performing output verification on the output data of the to-be-deployed model according to the output data format; and
determining that the output verification of the to-be-deployed model passes if a format of the output data is the same as the output data format.

4. The method of claim 1, wherein determining the inference service resource from the plurality of running environments and allocating the inference service resource to the to-be-deployed model comprises:
obtaining a file format of the to-be-deployed model, and converting the file format of the to-be-deployed model into a target defined format; and
determining a basic inference service resource required by the to-be-deployed model subject to format converting, determining the inference service resource from the plurality of running environments according to the basic inference service resource, and allocating the inference service resource to the to-be-deployed model subject to the format converting.

5. The method of claim 4, further comprising:
proceeding to determining the inference service resource from the plurality of running environments, to determine another inference service resource that is to be allocated to the to-be-deployed model, if the inference parameter value is less than the preset inference parameter threshold, wherein:
the plurality of running environments comprise running environments formed by changing at least one of number of graphics processing units (GPUs), models of the GPUs, or GPU running schemes.

6. The method of claim 1, wherein the to-be-deployed model is obtained by training a target training framework, and wherein the target training framework is one of Caffe, Caffeine2, TensorFlow, MxNet, CNTK, or Pytorch.

7. The method of claim 1, wherein training sample data for the to-be-deployed model comprises at least one of disease diagnosis and treatment information, personal healthcare information, or medical facility information.

8. A terminal device, comprising:
a processor; and
a memory coupled with the processor and configured to store computer programs, wherein the computer programs comprise program instructions, and the processor is configured to invoke the program instructions to:
obtain a to-be-deployed model and an input/output description file of the to-be-deployed model;
determine input data according to the input/output description file and perform output verification on the to-be-deployed model based on the input/output description file and the input data;
determine an inference service resource from a plurality of running environments and allocate the inference service resource to the to-be-deployed model, on condition that the output verification of the to-be-deployed model passes;
determine an inference parameter value of executing an inference service based on the inference service resource by the to-be-deployed model; and
generate a resource configuration file and an inference service interface of the to-be-deployed model according to the inference service resource to complete deployment of the to-be-deployed model, if the inference parameter value is greater than or equal to a preset inference parameter threshold.

9. The terminal device of claim 8, wherein the processor configured to invoke the program instructions to determine the input data according to the input/output description file is configured to invoke the program instructions to:
determine an input node and an input data format corresponding to the input node according to the input/output description file; and
generate the input data of the input node according to the input data format.

10. The terminal device of claim 9, wherein the processor configured to invoke the program instructions to perform the output verification on the to-be-deployed model based on the input/output description file and the input data is configured to invoke the program instructions to:
input the input data into the to-be-deployed model through the input node;
determine an output node and an output data format corresponding to the output node according to the input/output description file, and obtain output data of the to-be-deployed model from the output node;
perform output verification on the output data of the to-be-deployed model according to the output data format; and
determine that the output verification of the to-be-deployed model passes if a format of the output data is the same as the output data format.

11. The terminal device of claim 8, wherein the processor configured to invoke the program instructions to determine the inference service resource from the plurality of running environments and allocate the inference service resource to the to-be-deployed model is configured to invoke the program instructions to:
obtain a file format of the to-be-deployed model, and convert the file format of the to-be-deployed model into a target defined format; and
determine a basic inference service resource required by the to-be-deployed model subject to format converting, determine the inference service resource from the plurality of running environments according to the basic inference service resource, and allocate the inference service resource to the to-be-deployed model subject to the format converting.

12. The terminal device of claim 11, wherein the processor is further configured to invoke the program instructions to:
proceed to determining the inference service resource from the plurality of running environments, to determine another inference service resource that is to be allocated to the to-be-deployed model, if the inference parameter value is less than the preset inference parameter threshold, wherein:
the plurality of running environments comprise running environments formed by changing at least one of number of graphics processing units (GPUs), models of the GPUs, or GPU running schemes.

13. The terminal device of claim 8, wherein the to-be-deployed model is obtained by training a target training framework, and wherein the target training framework is one of Caffe, Caffeine2, TensorFlow, MxNet, CNTK, or Pytorch.

14. A non-transitory computer-readable storage medium storing computer programs, wherein the computer programs comprise program instructions which, when executed by a processor, cause the processor to:
obtain a to-be-deployed model and an input/output description file of the to-be-deployed model;
determine input data according to the input/output description file and perform output verification on the to-be-deployed model based on the input/output description file and the input data;
determine an inference service resource from a plurality of running environments and allocate the inference service resource to the to-be-deployed model, on condition that the output verification of the to-be-deployed model passes;
determine an inference parameter value of executing an inference service based on the inference service resource by the to-be-deployed model; and
generate a resource configuration file and an inference service interface of the to-be-deployed model according to the inference service resource to complete deployment of the to-be-deployed model, if the inference parameter value is greater than or equal to a preset inference parameter threshold.

15. The non-transitory computer-readable storage medium of claim 14, wherein the program instructions executed by the processor to determine the input data according to the input/output description file are executed by the processor to:
determine an input node and an input data format corresponding to the input node according to the input/output description file; and
generate the input data of the input node according to the input data format.

16. The non-transitory computer-readable storage medium of claim 15, wherein the program instructions executed by the processor to perform the output verification on the to-be-deployed model based on the input/output description file and the input data are executed by the processor to:
input the input data into the to-be-deployed model through the input node;
determine an output node and an output data format corresponding to the output node according to the input/output description file, and obtain output data of the to-be-deployed model from the output node;
perform output verification on the output data of the to-be-deployed model according to the output data format; and
determine that the output verification of the to-be-deployed model passes if a format of the output data is the same as the output data format.

17. The non-transitory computer-readable storage medium of claim 14, wherein the program instructions executed by the processor to determine the inference service resource from the plurality of running environments and allocate the inference service resource to the to-be-deployed model are executed by the processor to:
obtain a file format of the to-be-deployed model, and converting the file format of the to-be-deployed model into a target defined format; and
determine a basic inference service resource required by the to-be-deployed model subject to format converting, determine the inference service resource from the plurality of running environments according to the basic inference service resource, and allocate the inference service resource to the to-be-deployed model subject to the format converting.

18. The non-transitory computer-readable storage medium of claim 17, wherein the program instructions, when executed by the processor, further cause the processor to:
proceed to determining the inference service resource from the plurality of running environments, to determine another inference service resource that is to be allocated to the to-be-deployed model, if the inference parameter value is less than the preset inference parameter threshold, wherein:
the plurality of running environments comprise running environments formed by changing at least one of number of graphics processing units (GPUs), models of the GPUs, or GPU running schemes.

19. The non-transitory computer-readable storage medium of claim 14, wherein the to-be-deployed model is obtained by training a target training framework, and wherein the target training framework is one of Caffe, Caffeine2, TensorFlow, MxNet, CNTK, or Pytorch.

20. The non-transitory computer-readable storage medium of claim 15, wherein the to-be-deployed model is obtained by training a target training framework, and wherein the target training framework is one of Caffe, Caffeine2, TensorFlow, MxNet, CNTK, or Pytorch.
